(19) United States
(12) Patent Application Publication
(10) Pub. No.: US 2016/0031788 A1
(43) Pub. Date: Feb. 4, 2016

(54) METHOD OF MANUFACTURING DICARBOXYLIC ACIDS AND DERIVATIVES FROM COMPOSITIONS COMPRISING KETOCARBOXYLIC ACIDS

(71) Applicant: SEGETIS, INC., Golden Valley, MN (US)

(72) Inventors: ERICH J. MOLITOR, ELK RIVER, MN (US); BRIAN D. MULLEN, DELANO, MN (US)

(21) Appl. No.: 14/776,311

(22) PCT Filed: Mar. 11, 2014
(86) PCT No.: PCT/US14/23115

§ 371 Date: Sep. 14, 2015

Related U.S. Application Data
(60) Provisional application No. 61/798,129, filed on Mar. 15, 2013.

(51) Int. Cl. C07C 51/27; C07D 307/20

(52) U.S. Cl. CPC C07C 51/27; C07D 307/20

(57) ABSTRACT

A crude composition comprising a ketocarboxylic acid such as levulinic acid, derived from biomass in an aqueous phase comprising impurities, can be efficiently converted in high yield to the corresponding dicarboxylic acid, such as succinic acid, which can be purified and dried.

### PRIORITY

This application claims priority to U.S. Provisional Patent Application Ser. No. 61/798,129, filed Mar. 15, 2013, the contents of which are incorporated herein by reference in their entirety for all purposes.

### BACKGROUND

This disclosure relates to the manufacture of dicarboxylic acids from ketocarboxylic acids. In particular, this disclosure relates to the manufacture of dicarboxylic acids from the impure aqueous compositions, derived from biomass, comprising ketocarboxylic acids.

### SUMMARY

The inventors hereof have discovered a method for advantageously converting a crude composition comprising a ketocarboxylic acid such as levulinic acid, derived from biomass and having significant levels of impurities, to a dicarboxylic acid, such as succinic acid. Crude products can be contaminated, for example, with by-products from the manufacture of the ketocarboxylic acids, for example, by hydrolytic decomposition of furfuryl alcohol, sugars, starch, ligno-cellulose or cellulose.

In one embodiment, a method for producing a dicarboxylic acid, such as succinic acid, from biomass comprises obtaining from biomass a crude composition comprising, along with at least 1.0 wt. % of impurities comprising biomass residues, a ketocarboxylic acid, such as levulinic acid, having the following structure

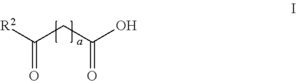

wherein R² is C₁-C₆ alkyl and a=0-3, specifically, 1-3 and more specifically, 1-2; reacting the crude composition to oxidize the ketocarboxylic acid in the presence of an oxidizing agent and optionally a catalyst, to produce the corresponding dicarboxylic acid; and optionally purifying and drying the dicarboxylic acid to obtain a solid composition comprising greater than 95 wt. % of dicarboxylic acid, specifically, in which the R² is replaced by an OH.

In one embodiment, the invention is directed to a method for the production of succinic acid or succinic anhydride from biomass comprise obtaining a crude composition comprising levulinic acid from biomass and converting the levulinic acid in the composition into succinic acid or succinic anhydride by a process as defined above.

In one embodiment, the invention is directed to a method for producing succinic acid from biomass comprising obtaining from biomass, in the presence of an acid catalyst, a hydrolysate that is a crude composition comprising, along with at least 1.0 wt. % of impurities comprising biomass residues and at last 5 wt. % water, levulinic acid having the following structure:

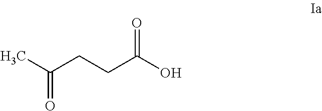

thereafter heating the crude composition to oxidize the levulinic acid in the presence of an oxidizing agent to produce succinic acid, wherein the oxidizing agent is nitric acid, and optionally a catalyst comprising vanadium pentoxide, optionally in the further presence of a metallic nitrite, wherein the crude composition is heated in a continuous reaction system to a temperature in the range of from 10° C. to 140° C., more specifically, from 20° C. to 100° C., more specifically, from 20° C. to 80° C., for less than ten, nine, eight, seven, six, five, four, three, two hours or even one hour, followed by quenching, in a continuous reaction system; and then optionally purifying and drying the succinic acid to obtain a solid composition comprising at least 95 wt. % succinic acid.

### BRIEF DESCRIPTION OF THE DRAWINGS

FIG. 1 is a Size Exclusion Chromatogram (UV detector, 1=300 nm) of soluble oligomers in material I, and Examples 6-8.

### DETAILED DESCRIPTION

Dicarboxylic acids such as succinic acid can be produced from biomass. Specifically, succinic acid can be produced from levulinic acid which in turn can be obtained from biomass. However, such processes, starting with biomass, are not fully selective towards the formation of desired products and, depending on the process, various impurities can be formed during the process.

Furthermore, such processes frequently require the necessity to work under diluted aqueous conditions. These conditions can impose significant challenges on the down-stream processing of the reaction products. It is therefore desirable to provide a suitable synthetic route for the production of succinic acid from biomass via levulinic acid (4-oxopentanoic acid), which can be obtained from biomass.

Various methods for producing levulinic acid and its derivatives from biomass are known, which results in impure aqueous compositions comprising levulinic acid or its derivatives. For example, levulinic acid can be obtained by an acid catalyzed conversion of low cost (hemi)cellulosic material. Also, for example, U.S. Pat. No. 6,054,611 discloses the production of levulinic acid as dehydration products of 5-carbon or 6-carbon sugars, along with by-products such as furfural, 5-HMF (5-hydroxymethyl-2-furaldehyde), succinic acid, maleic acid, or fumaric acid.

Succinic acid can be used for a multitude of biobased products. For example, it can serve as a platform chemical for the production of chemicals like succinic anhydride, 1,4-butanediol, THF (tetrahydrofuran), γ-butyrolactone, 2-pyrrolidone, succinate esters and the like. In addition, succinic acid and its derivatives have potential for use as building blocks in the manufacture of various polymers.

Once levulinic acid is obtained, succinic acid can be produced. For example, U.S. Pat. No. 2,676,186 discloses a process for the synthesis of succinic acid from levulinic acid in which ammonium metavanadate catalyst at elevated temperatures, for example, 275-400° C., is used for converting levulinic acid into succinic acid. More recently, US 2012/044168 discloses the synthesis of succinic acid by a chemical route of converting levulinic acid into succinic acid, which process comprises (a) obtaining levulinic acid from biomass, and (b) heating levulinic acid, notably at mild temperatures, in the presence of nitric acid.

The latter patents have serious drawbacks. U.S. Pat. No. 2,676,186 requires very high temperatures above 200° C., involving oxidation of levulinic acid in vapor form. Thus, it can be assumed that the levulinic acid must be in pure form. US 2012/044168 discloses a process of preparing succinic acid from levulinic that uses levulinic acid in either pure form or from biomass. A batch system is used in which levulinic acid is heated, in the examples, for 1 hour to 4 hours. Examples 9 and 11 of US 2012/044168 use levulinic acid obtained from biomass, i.e., D-fructose (Example 8) and cellulose (Example 9), respectively. In both cases, considerable char is apparently formed during the production of levulinic acid. The crude levulinic acid, as a black liquid or suspension, was dried and then converted to succinic acid.

It would be desirable to efficiently convert a ketocarboxylic acid such as levulinic acid, derived from biomass, to a dicarboxylic acid such as succinic acid in a process, such as a continuous process, without having to remove water from, or dry, the levulinic acid, wherein char is reduced or eliminated in forming the levulinic acid and/or removed before conversion of the levulinic acid to succinic acid.

Commercially available ketocarboxylic acids that are primarily by-products from the acid-catalyzed degradation of biomass such as furfuryl alcohol, sugars, starch, ligno-cellulose or cellulose can be contaminated with impurities. Such impurities can include solids, angelica lactones, formic acid, furanics, aldehydes, and various oligomers. Levulinic acid, a specific ketocarboxylic acid, can be derived directly from the acidic hydrolytic degradation of various biomass feedstocks and, thus, can contain water and acid catalyst or only water. The crude levulinic-acid-containing product stream can also be obtained from various stages of solids filtration, extraction, and or purification from the acidic hydrolysis mixture and thus contain various amounts of extraction solvent. Additionally, the crude levulinic acid could be essentially free of water, acid, solids, or extraction solvent but not yet be further refined. For example, the levulinic acid impurities could contain various unknown oligomeric species (such as those that may be characterized by size exclusion chromatography).

In the present process, the crude ketocarboxylic acid (e.g., levulinic acid) stream can be converted to a crude dicarboxylic acid stream (e.g., succinic acid stream) by treatment under process conditions which can include nitric acid (optionally with sodium nitrite) and optionally vanadium(V) oxide to oxidize the levulinic acid as follows.

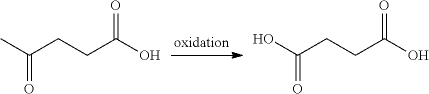

In one aspect, crude ketocarboxylic acid such as levulinic acid, containing impurities, derived from biomass, can be efficiently converted directly from a crude composition comprising a ketocarboxylic acid, such as levulinic acid, that is, a biomass-derived hydrolysate. The corresponding dicarboxylic acids can be obtained from the crude composition without removing impurities, or water and impurities, from the hydrolysate composition comprising ketocarboxylic acid such as levulinic acid. Furthermore, the ketocarboxylic acid, such as levulinic acid, can be converted to the dicarboxylic acid in a continuous, semi-continuous or batch process under heat, by appropriate use of time and temperature to obtain a composition comprising the dicarboxylic acid. Specifically, a continuous conversion of crude ketocarboxylic acid, such as levulinic acid, to diacid such, as succinic acid, can be accomplished by contacting crude ketocarboxylic acid, such as levulinic acid, with an oxidant and optionally a catalyst, such as a mixture of nitric acid (optionally with a metallic nitrite such as sodium nitrite) and optionally vanadium (V) oxide. The reaction can be allowed to react at a pre-selected temperature for a limited short period of time before quenching, for example, by cooling. The reaction product can then be subjected to purification and drying to obtain solid succinic acid in the form of a white powder or crystals. After conversion to the dicarboxylic acid, the dicarboxylic acid can be purified and used for other synthetic transformations. Thus, dicarboxylic acids can be efficiently produced from crude ketocarboxylic acid, such as levulinic acid, and then obtained in high purity without producing and purifying or drying the ketocarboxylic acids. In particular, the ketocarboxylic acid, such as levulinic acid, can contain significant amounts of oligomeric impurities, aldehydes, formic acid, sulfur-containing acid impurities, transition metal impurities, or the like, prior to oxidation to the corresponding dicarboxylic acid. The crude ketocarboxylic acid, such as levulinic acid, can have significant color. However, charring can be significantly reduced or eliminated by appropriate control of temperature applied to the ketocarboxylic acid, such as levulinic acid, or concentration of mineral acid impurities, for a relatively limited period of time in a continuous, semi-continuous or batch process. Char that is produced in the form of particles can be readily removed, for example, by filtration. In most cases, the significant dark color of the crude reaction mixture containing the ketocarboxylic acis, such as levulinic acid, can be lessened to a significant extent after oxidation.

The term “biomass,” as used herein, includes sludges from paper manufacturing processes; agricultural residues; bagasse pity; bagasse; molasses; aqueous oak wood extracts; rice hull; oats residues; wood sugar slops; fir sawdust; corncob furfural residue; cotton balls; raw wood flour; rice; straw; soybean skin; soybean oil residue; corn husks; cotton stems; cottonseed hulls; starch; potatoes; sweet potatoes; lactose; sunflower seed husks; sugar; corn syrup; hemp; waste paper; wastepaper fibers; sawdust; wood; residue from agriculture or forestry; organic components of municipal and industrial wastes; waste plant materials from hard wood or beech bark; fiberboard industry waste water; post-fermentation liquor; furfural still residues; sugars, including C6 sugars; lignocellulos and cellulose; starch; and combinations thereof. Thus biomass can include polysaccharides, disaccharides, and monosaccharides.

The ketocarboxylic acid such as levulinic acid can be obtained by the hydrolysis of biomass products in the presence of various acid catalysts. Acid catalysts can be either a Lewis or Brønsted-Lowry acid. Suitable Brønsted acids used to convert the biomass materials described herein, for example sugars, can include mineral acids, such as but not limited, to sulfuric acid, hydrochloric acid, hydrobromic acid, hydroiodic acid, hydrofluoric acid, perchloric acid and mixtures thereof. Acid catalysts that are known homogeneous catalysts for ketal formation can be used, for example strong protic acid catalysts, e.g., Brønsted-Lowry acids that have a Kₐ of 55 or greater. Examples of strong protic acid catalysts include sulfuric acid, arylsulfonic acids, and hydrates thereof such as p-toluenesulfonic acid monohydrate, methane sulfonic acid, camphor sulfonic acid, dodecyl benzene sulfonic acid, perchloric acid, hydrobromic acid, hydrochloric acid, 2-naphthalene sulfonic acid, and 3-naphthalene sulfonic acid. In other embodiments, weak protic acid catalysts, e.g., having a Kₐ of less than 55, can be used, for example phosphoric acid, orthophosphoric acid, polyphosphoric acid, and sulfamic acid. Aprotic (Lewis acid) catalysts can include, for example, titanium tetraalkoxides, aluminum trialkoxides, tin II alkoxides, carboxylates, organo-tin alkoxides, organo-tin carboxylates, and boron trifluoride. A combination comprising any one or more of the foregoing acid catalysts can be used.

Instead of, or in addition to the homogenous acid catalyst, a heterogeneous acid catalyst can be used, where the acid catalyst is incorporated into, onto, or covalently bound to, a solid support material such as resin beads, membranes, porous carbon particles, zeolite materials, and other solid supports. Many commercially available resin-based acid catalysts are sold as ion exchange resins. One type of useful ion exchange resin is a sulfonated polystyrene/divinyl benzene resin, which supplies active sulfonic acid groups. Other commercial ion exchange resins include LEWATIT® ion exchange resins sold by the Lanxess Company of Pittsburgh, Pa.; DOWEX™ ion exchange resins sold by the Dow Company of Midland, Mich.; and AMBERLITE® and AMBERLYST® ion exchange resins sold by the Dow Company of Midland, Mich. In embodiments, AMBERLYST® 15, AMBERLYST® 35, AMBERLYST® 70 are used. In these embodiments, the resin-based catalyst is washed with water, and subsequently, an alcohol, such as methanol or ethanol, and then dried prior to use. Alternatively, the resin is not washed before its first use. In embodiments, Nafion® resins (from DuPont in Wilmington, Del.) can also be used as heterogeneous catalysts in neat form or filled with silica. In use, the heterogenous catalysts are added to a reaction mixture, thereby providing a nonvolatile source of acid protons for catalyzing the reactions. The heterogenous catalysts can be packed into columns and the reactions carried out therein. As the reagents elute through the column, the reaction is catalyzed and the eluted products are free of acid. In other embodiments, the heterogenous catalyst is slurried in a pot containing the reagents, the reaction is carried out, and the resulting reaction products filtered or distilled directly from the resin, leaving an acid-free material.

In an embodiment, this process uses a high concentration of sulfuric acid, such as about 1 to 80 wt %, specifically about 10 to about 60 wt %, and more specifically 20-55 wt %, relative to the total weight of the reactants, which has several distinct advantages. For one, the reactions can be run at lower temperatures compared to low acid processes and still hydrolyze the sugars in a reasonable time frame. It has been discovered that under these high acid, low-temperature reaction conditions (e.g., 60-160° C., specifically 80-150° C., and more specifically 90-140° C.), the char by-product that is formed is in the form of suspended particles that are easier to remove from the reactor and that can be filtered from the liquid hydrolysate product stream. In contrast, with low acid conditions, high temperature is required to effectively hydrolyze the sugar in a reasonable time frame and those conditions produce a char by-product that coats the reactor components in such a manner that it can be difficult to remove without mechanical or hydraulic energy and, for the most part, does not stay suspended in the reaction mixture. As mentioned above the biosourced hydrolysate comprising the ketocarboxylic acid such as levulinic acid can be contaminated with a variety of by-products arising from their production. A crude ketocarboxylic acid such as levulinic acid composition can comprise, for example, 1-10 wt. %, or 2-8 wt. % of contaminants. Furthermore, the present method can effectively use crude ketocarboxylic acid such as levulinic acid compositions that comprise undried or wet biosourced products, that is, containing in some embodiments 5 wt. %, 10 wt. %, 30 wt. %, or up to 50 wt. % of water and in other embodiments 5-95 wt. %, 10-90 wt. % water, or 20-60 wt. % water.

Contaminants that may be present in total amounts of greater than about 1 wt. %, can more specifically include one or more biomass residues, specifically 0 to 10 wt. % or more of furfural, formic acid, furfuryl alcohol, hydroxymethyl furfural, angelica lactone, acetic acid, solid humins, lignin, methanol, glucose, fructose, unknown oligomers, solid char particles and the like. Such residues of biomass can arise from the manufacture of the ketocarboxylic acids from biomass such as sugars, cellulose, lignocellulose, or other polysaccharides such as starches, inulin, and xylan.

The following examples illustrate compositions of commercially available crude levulinic acid. In one sample of a commercial product, the commercially available crude levulinic acid (LA) is 94% pure and contains greater than 0.5 wt. % low molecular weight components (sulfuric acid, angelica lactones, furanics, extraction solvent) and greater than 2 wt. % higher molecular weight impurities (oligomers of unknown composition). A second sample of crude levulinic acid contains greater than 10 wt. % water, greater than 10 wt. % H₂SO₄, less than 1 wt. % sodium salts, greater than 5 wt. % levulinic acid, less than 0.5 wt. % angelica lactone and furanic impurities, and less than 0.5 wt. % solid humins of unknown composition. The yellowness index (YI) of the crude levulinic acid is greater than 50. The sample is dark yellow to black in color.

In the present process, pure succinic acid can be produced from aforementioned examples of crude levulinic acid that contains a significant amount of impurities. The resulting pure succinic acid can contain less than 5 wt. % impurities, specifically less than 1 wt. % impurities, and more specifically less than 0.1 wt. % impurities, based on the total weight of the succinic acid.

The ketocarboxylic acid for use in making the corresponding dicarboxylic acid, need not be subjected to esterification, crystallization, or isolation by various purification methods. Methods of isolating or purifying a ketocarboxylic ester to a limited extent can include washing, crystallizing, filtering, liquid-liquid phase extraction, separating, precipitation, adsorption, or a combination of at least one of the foregoing. In an embodiment, however, such methods are avoided or excluded, in order to provide a low cost method of manufacture, by purifying the product at a later stage, including after the production of succinic acid by oxidation of the ketocarboxylic acid. Thus, a crude ketocarboxylic hydrolysate from biomass can be used for conversion to the corresponding dicarboxylic acid directly, or after filtering char, specifically while remaining in an aqueous phase. In one advantageous aspect, the conversion can take place continuously or continuously in the same reactor in which the hydrolysate comprising the levulinic acid is obtained.

In an embodiment, a biosourced crude composition comprises the following ketocarboxylic acid:

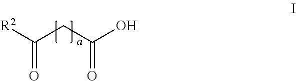

wherein R² is C₁-C₆ alkyl and a=0-3. In another specific embodiment, with reference to formula I, R² is C₁-C₆ alkyl and a=1-3. In another specific embodiment, with reference to formula I, R² is C₁-C₃ alkyl and a=1-2. In yet another specific embodiment, with reference to formula I, R² is methyl and a=1 or 2, specifically R² is methyl and a=2.

In a more specific embodiment, the starting composition for the oxidation to the corresponding dicarboxylic acid comprises levulinic acid represented by the following structure:

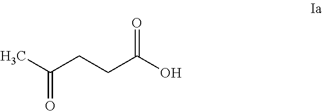

The starting composition further contains impurities or impurities and water. As shown in Reaction I below, for example, crude levulinic acid (a levulinic acid I wherein a=2) can be reacted under heat with an oxidizing agent such as nitric acid to form the dicarboxylic acid.

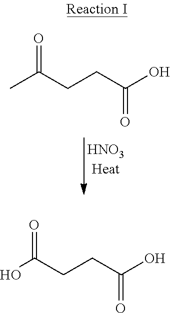

In the present process, an oxidant, such as nitric acid and optionally a catalyst, such as a catalyst comprising vanadium oxide can be used in an amount effective to establish the formation of succinic acid in good yield. In other embodiments, combinations of vanadium and copper catalysts may also be used. In one case, when using nitric acid, this amount can involve more than 0.1 equivalent of nitric acid calculated based on the amount of levulinic acid. Specifically, more than 0.5 equivalent and most specifically a stoichiometric or excess amount of nitric acid, with respect to levulinic acid or other ketocarboxylic acid, can be used. The nitric acid can be present in an amount of at least 100 gram/liter. In an embodiment, the nitric acid is present in excess and has a concentration of at least 200 gram/liter.

Suitable oxidants to transform the ketocarboxylic acid into a dicarboxylic acid include, for example, a permanganate, hypochlorite, oxygen, ozone, OXONE®, nitric acid, nitric oxide, sodium nitrite, a peroxide, such as hydrogen peroxide, as well as others described herein.

Suitable metal containing catalysts that can be used in combination with the oxidants contain for example, platinum, palladium, ruthenium, copper, cobalt, vanadium, tungsten, iron, silver, manganese, or gold. Specific catalysts may include, but are not limited to MeReO₃ (methyltrioxorhenium (VII)), RuCl₃, polyoxometalates, such as [AlMn^{II/III}(OH₂)W₁₁O₃₉]^{6-/7} copper compounds, such as copper sulfate or copper oxide, cobalt compounds, platinum catalysts such as supported platinum or complexes, Perovskite-type complexes (LaMnO₃), metal bromide catalysts, such as Co—Mn—Br, or Au/TiO₂.

In an embodiment, the production of succinic acid from levulinic acid in the presence of an oxidizing agent such as nitric acid is conducted at elevated temperature. Although higher heating temperatures and longer times are possible, it will be clear that the advantages of this process will be most pronounced if the heating is conducted at relatively mild elevated temperatures and relatively shorter times, specifically with quenching. This can help to avoid or limit charring. The heating refers to a temperature above ambient temperature (generally above 18° C.), optionally at atmospheric pressure. It will be understood by the skilled person that lower temperatures can be used, and would be regarded “elevated,” if the reaction is conducted under elevated (i.e. above atmospheric) pressure. In an embodiment, the reaction temperature is in the range of from 10° C. to 140° C., more specifically, from 20° C. to 100° C., more specifically, from 20° C. to 80° C. In an embodiment, the reaction temperature, calculated on the basis of atmospheric pressure, is below 100° C., for example, the reaction is conducted at a temperature between 20° C. and 80° C. and more specifically between 30° C. and 70° C.

It will be understood that the reaction of the crude hydrolysate comprising levulinic acid is conducted for a sufficient period of time, such as, for less than ten, nine, eight, seven, six, five, four, three, two hours or even one hour, specifically prior to quenching, in order to obtain the reaction product in good yield Specifically, in an embodiment, the reaction can be conducted for less than one hour, specifically for one minute to 45 minutes, specifically 5 minutes to 40 minutes, more specifically 10 minutes to 30 minutes. The reaction can advantageously be conducted on a continuous basis and can include recycle streams containing levulinic acid. One of ordinary skill in the art can select the reaction conditions (pressure, temperature, time) and reaction equipment that is best applicable to a given starting material or hydrolysate comprising impure ketocarboxylic acid. Depending on the particular composition of the crude feedstock, the concentration of oxidizing agent, catalyst, residence time, temperature and other conditions can be pre-selected or varied to obtain good yields of succinic acid or similar dicarboxylic acid at low overall cost, especially compared to a batch process or the use of pure levulinic acid obtained from biomass.

In one embodiment, Nitric acid can be used as an oxidizing agent in the conversion of levulinic acid into succinic acid, with the formation of CO or CO₂. Without wishing to be bound by theory, it is believed that the nitric acid not only acts as an oxidative agent, but also as a catalyst or co-catalyst for the conversion, which allows the relatively mild reaction conditions in obtaining good yields.

In an embodiment, nitric acid is used in as an oxidative agent in the chemical conversion of levulinic acid into succinic acid. The nitric acid can be used in a concentration of at least 200 g/liter, specifically in aqueous solution at 200 to 650 g/liter. In addition to nitric acid, sodium nitrite (NaNO₂) is optionally present. Small amounts of sodium nitrite or the like (0.1 to 0.5% by weight compared to nitric acid) can serve to accelerate the reaction.

The term nitric acid can also include the usual nitrogen oxides such as NO and NO₂ that are present in nitric acid. In addition to nitric acid, other acids and/or oxidizing agents can be present. Advantageously, the nitric acid can further oxidize unstable components into, for example, gaseous products that can be readily removed. The succinic acid that is formed is stable under reaction conditions applied, which can lead to a much simpler and improved purification or other down-stream processing.

Optionally, in the conversion of a ketocarboxylic acid, specifically levulinic acid to a dicarboxylic acid, specifically succinic acid, a solid catalyst, such as those described herein, can be used to promote the reaction. Specifically, the conversion reaction can be advantageously conducted in the presence of vanadium pentoxide as a catalyst, optionally in combination with the presence of nitric acid and optional metal nitrite. Vanadium pentoxide (V₂O₅) can be used in a catalytic amount, specifically 0.01-2.0 wt. %. Such solid catalyst can be advantageously used to increase the rate and the selectivity of the reaction.

The reaction process, with or without vanadium pentoxide as a catalyst, can be conducted in any reaction equipment normally used for such chemical processes involving acidic materials. Specifically, equipment can be used that can sustain the oxidative properties of nitric acid.

Advantageously, biomass is used for the production of succinic acid from levulinic acid. Specifically, levulinic acid is used as a starting material, which can be obtained from such biomass. Advantageously, the levulinic acid can be transformed into succinic acid with limited or no purification, in a continuous, semi-continuous or batch process, specifically a continuous process, without removing water from the levulinic acid. Biomass is an advantageous source of materials, since it can be available on a renewable basis, including dedicated energy crops and trees, agricultural food and fee crop residues, (recycled) paper residues, aquatic plants, animal, and other wastes, as indicated by the definition of biomass.

In this regard, an aspect is directed to a method for the production of succinic acid from biomass comprising (a) obtaining a crude composition comprising levulinic acid as an acid hydrolysate from biomass, and (b) subjecting the crude composition, containing impurities, optionally without dewatering or isolation, to elevated temperature in the presence of an oxidizing agent in a continuous process. The oxidizing agent can comprise nitric acid, optionally with a metallic nitrite, and/or vanadium oxide and other materials as described herein.

It will be understood that, in an overall process, not only succinic acid can be readily produced, but also carboxylic acid derivatives thereof. Such derivatives can include, but are not limited to, succinic anhydride, succinic esters, and succinic amides. Generally, these carboxylic acid derivatives can be produced as disclosed herein from the corresponding carboxylic acid derivatives of levulinic acid. Esters can be made up of any alcohol, e.g. C₁₋₃₀ alcohol, specifically C₁₋₁₀ alcohol, and more specifically C₁₋₆ alcohol. For amides, the analogous amines can be employed.

The conversion of levulinic acid into succinic acid, which can be carried out in near quantitative conversion yield (generally over 90% conversion, and particularly capable of over 95% conversion, typically approximately 99%) also may involve the co-production of CO and/or CO₂. One of ordinary skill will be able to use a standard chemical work-up in separating, isolating, and purifying the levulinic acid obtained in the desired product.

The conversion of crude levulinic acid into succinic acid may also produce acetic acid or other low molecular weight acids.

The conversion of crude levulinic acid into succinic acid may also oxidize higher molecular weight oligomeric impurities into lower molecular weight compounds, comprising succinic acid and acetic acid. The reduction in oligomeric content in the crude reaction mixture may be monitored by size exclusion chromatography (SEC).

The oxidizing agent, for example nitric acid, can be charged directly into the composition comprising the levulinic acid or alternatively it can be diluted in water prior to being charged into the reactant mixture. Dilute nitric acid can be continuously added to the reactant mixture throughout the course of the reaction or alternatively it can be added instantaneously to the reactant mixture in a single charge.

In an embodiment, the reaction to produce the dicarboxylic acid can be conducted in a continuous reactor or in a semi-continuous reactor. It is desirable for the reactor to have heating, cooling, agitation, condensation, and distillation facilities.

In an embodiment, a system (not shown) for producing the dicarboxylic acid can comprise a single continuous stirred tank reactor that is fitted with a distillation column. The distillation column can be used to remove excess by-products, impurities, and water from the reaction.

In a continuous reactor system, the reactants are charged to a first reactor. When the conversion of reactants to products is measured to be greater than or equal to about 50%, a portion of the product mixture from the first reactor can be subjected to additional finishing processes in a second reactor, while at the same time additional reactants and catalyst are continuously being charged to the first reactor to be converted into the ketocarboxylic acid. A continuous reactor system can employ a plurality of reactors in series or in parallel so that various parts of the process can be conducted in different reactors simultaneously. In an embodiment, the same reactor can be used for both production of the hydrolysate comprising the levulinic acid and its oxidative conversion to the dicarboxylic acid.

In an embodiment, the reactor comprises a plurality of reactors (e.g., a multistage reactor system) that are in fluid communication with one another in series or in parallel. The plurality of reactors can be used to react the levulinic acid to obtain the corresponding dicarboxylic acid, to recycle the reactants, and to remove unwanted byproducts and impurities so as to obtain a dicarboxylic acid that is pure and stable. In an embodiment, a portion of the plurality of reactors can be used primarily to react reactants to manufacture the dicarboxylic acid, while another portion of the plurality of reactors can be used primarily to isolate the dicarboxylic acid and yet another portion of the plurality of reactors can be used to remove the residual catalyst and other byproducts that can hamper the formation of a stable product that has good shelf stability.

Optionally, the reaction can be carried out under a blanket of an inert gas (e.g., argon, nitrogen, and the like) or alternatively can be carried out under pressure. A reactor can be subjected to a pressure of 1 to about 2000 psi, specifically about 10 to about 500 psi.

Upon completion of the reaction, the solution can be cooled down followed by purification of the dicarboxylic acid. The crystalline dicarboxylic acid can then be washed in a first solvent to remove any contaminants. The washed dicarboxylic acid can then be re-dissolved in a second solvent and recrystallized to produce a pure form of the dicarboxylic acid. The first and the second solvent can be the same or different. In an embodiment, the first solvent is water and the second solvent is water as well. Water also removes color bodies, acid catalyst, and unreacted levulinic acid from the reaction mixture. The water can contain some salt or dilute base. Suitable heating and cooling steps can be performed to conduct re-crystallization.

While water can be used for purification, organic solvents can also be used to crystallize it. Examples of other organic solvents are methanol, ethanol, acetone, benzene, toluene, diethyl ether, ethyl acetate, or the like, or a combination comprising at least one of the foregoing solvents, using a purification scheme that can be conventionally practiced by one of ordinary skill in the art.

In another embodiment, after the conversion of levulinic acid to dicarboxylic acid, a base can be added to the mixture to precipitate the dicarboxylate salt. An exemplary additional base is sodium hydroxide. Other bases conventionally known can also be used. The base can be added in an amount of about 0.8 to about 1.5 moles, specifically about 0.9 to about 1.3 moles, based on the total moles of the dicarboxylic acid. Other anions for the dicarboxylate salt are lithium, sodium, potassium, calcium, magnesium, ammonium and NHₙR₄₋ₙ, wherein R=alkyl, aryl, alkaryl, or arylalkyl and where n=0-4.

Solid impurities can be filtered from the dicarboxylic acid product via a pressurized filter. The dicarboxylic salt can be re-acidified to form the dicarboxylic acid. In an embodiment, the acid used for re-acidification of the dicarboxylate salt is sulfuric acid. The sulfuric acid can be diluted with water prior to re-acidification. Other acids mentioned above can also be used.

In an embodiment, a filtered mixture of the dicarboxylic acid can be heated to remove the water. In order to remove the water, the filtered mixture can be heated to a temperature around the boiling point of water specifically about 80 to about 100° C., with or without vacuum. It can then be subjected to distillation to strip off organic impurities. In an embodiment, the distillation is vacuum distillation, conducted at a vacuum of about 1 to about 10 Torr, specifically about 3 to about 7 Torr.

It is desirable for the dicarboxylic acid to be of high purity. A pure form of the dicarboxylic acid that can be obtained by the present method has a purity of greater than or equal to about 95%, specifically greater than or equal to about 98%, and more specifically greater than or equal to about 99%, on a weight basis, while displaying a yellowness index of less than 20 units as measured by ASTM E 313, preferably less than 5 units as measured by ASTM E 313. The pure form of the dicarboxylic acid can comprise white powder and/or can comprise white shiny crystals.

The invention is further illustrated by the following non-limiting examples.

### EXAMPLES

### Methods

High Pressure Liquid Chromatography for Chemical Analysis:

Analysis of the product compositions were analyzed by High Pressure Liquid Chromatography (HPLC) using one of three methods as indicated in the examples.

The first HPLC method (method 1) uses a Waters® LC System (from Waters Corp. of Milford, Mass.) with a PDA 2998 Photodiode Array. The column is a Hamilton® X300 7 μm particle size, 250×4.1 mm column, the flow is isocratic at 2.0 mL/min, the sample temperature target is 25.0° C., the column temperature target is 50.0° C., and the mobile phase is 20% methanol and 80%, 20 mN phosphoric acid.

The second HPLC method (method 2) uses a Waters® LC 2695 System (from Waters Corp. of Milford, Mass.) with RI 2414 Differential Refractometer. The column is a Bio-Rad Aminex® HPX-87H, 300×7.8 mm column, the flow is isocratic at 0.60 mL/min, the sample temperature target is 25.0° C., the column temperature target is 50.0° C., and the mobile phase is 20 mM phosphoric acid in deionized water with 3% acetonitrile

The third HPLC method (method 3) uses a Waters® LC 2695 System (from Waters Corp. of Milford, Mass.) with RI 2414 Differential Refractometer. The column is a Supelcosil® LC-NH2, the flow is isocratic at 1.0 mL/min, the sample temperature target is 25.0° C., the column temperature target is 50.0° C., and the mobile phase is 80% acetonitrile and 20% water, 20 mM phosphoric acid.

Moisture Analyzer:

A Mettler Toledo® HG63 Halogen Moisture Analyzer with a drying temperature of 125° C. was used.

Char Washing Method:

Unless otherwise specified, the following char washing method was used. The char was placed in a Buchner funnel and first washed with 2×250 mL deionized water. A spatula was used to break up the char cake so that it was fully dispersed in the water on the Buchner funnel. After the water wash, the char was washed with 250 mL of acetone.

### Example 1

### Synthesis of Levulinic Acid (LA)

Into a beaker containing a magnetic stir bar was charged 43.25 g (0.24 mol) fructose and 50.08 g deionized water. The beaker was placed on a stir plate to dissolve the fructose. Into a 500 mL four neck round bottom flask containing a magnetic stir bar was charged 11.84 g deionized water and 153.35 g (1.00 mol) 64% sulfuric acid. The round bottom flask was situated in a heating mantle and equipped with a thermocouple, condenser, glass stopper and syringe pump tube. The sulfuric acid and water mixture was heated to 90° C. while stiffing at a rate of 650 RPM. Once the fructose was all dissolved it was charged into two 60 mL syringes and situated into a syringe pump. Once the acid and water mixture was up to temperature the addition of fructose solution was started via the syringe pump. The fructose solution was added over a course of 2.5 hours at a rate of 30.8 mL/hr. The concentration of fructose did not exceed 0.7%, and the concentration of 5-hydroxymethyl-2-furaldehyde (HMF) did not exceed 0.4% during the monosaccharide addition into the reactor. After all of the fructose had been added, the reaction was left to react for an additional hour and then was shut down and allowed to cool to ambient temperature. The solids that were formed remained suspended in the reactor during the entire reaction. Once the reaction mixture was cool it was filtered through a glass microfiber 1.1 μm filter paper. The solids were then washed with DI water and acetone. The moisture analyzer was used to determine the amount of solids in the reaction mixture. The results of the experiment showed 57.91 mol % yield of LA, 68.01 mol % yield of formic acid (FA) and an LA to char ratio of 2.18 using HPLC method 2.

### Example 2

### Synthesis of Levulinic Acid (LA)

The same procedure was followed as in Example 1 with different charged weights, and all of the fructose was added over 1.25 hours. Amounts were 38.03 g (0.21 mol) fructose and 25.60 g deionized water, 102.57 g deionized water and 103.04 g (1.05 mol) sulfuric acid. The syringe pump was set to 37.6 mL/hr. The concentration of fructose did not exceed 1.5 wt. %, and the concentration of HMF did not exceed 0.7 wt. % during the monosaccharide addition into the reactor. The solids that were formed remained suspended in the reactor during the entire reaction. The results of the experiment showed 81.37 mol % yield of LA, 95.03 mol % yield of FA and an LA to char ratio of 3.48 using HPLC methods 1 and 3.

### Example 3

### Synthesis of Succinic Acid

The product composition of Example 1 is filtered via a Buchner funnel, and then the filtrate is extracted with 1 L of solvent, LBX-98 (Merisol, Inc.). The solvent and formic acid are removed by distillation providing a crude mixture of greater than 80% purity of levulinic acid. Into 20 grams of this crude product is added 0.2 g V₂O₅, and the mixture is heated for 2 h at 120° C. to afford succinic acid.

### Example 4

### Synthesis of Succinic Acid

The product composition of Example 2 is filtered via a Buchner funnel, and then 150 g of filtrate is placed into a 3-necked flask with a condenser. Then 1 gram of concentrated HNO₃ is slowly added into the mixture and the mixture is allowed to react at 100° C. for 2 h to afford succinic acid.

### Example 5

### Synthesis of Succinic Acid

The product composition of Example 2 is filtered via a Buchner funnel, then filtrate is pumped into a continuous catalyst bed containing supported V₂O₅ (such as V-0501S or V-0701T from Engelhard now BASF). A solution of HNO₃ is also continuously added to the continuous reactor and contacted with the filtrate solution at the entry of the catalyst bed. The catalyst bed is maintained in a temperature between 20 and 100° C. and the residence time of the filtrate solution in the bed is from 10 seconds to 10 minutes. The reactor effluent is cooled rapidly to afford succinic acid.

### Examples 6-8

### Description of Analytical Methods

The HPLC method for Examples 6-8 used a Waters® LC 2695 System (from Waters Corp. of Milford, Mass.) with RI 2414 Differential Refractometer. The column was a Bio-Rad Aminex® HPX-87H, 300×7.8 mm column, the flow was isocratic at 0.60 mL/min, the sample temperature target was 25.0° C., the column temperature target was 50.0° C., and the mobile phase was 20 mM phosphoric acid in deionized water with 3% acetonitrile.

LC-MS analyses were performed using a Waters 2695 Alliance Separation Module connected to a Waters 996 PDA (photodiode array) UV detector and a Waters Micromass ZQ mass spectrometer. Conditions for the analysis of succinic acid and levulinic acid were as follows:

Column: Ascentis Express C-18 150×4.6 mm 2.7 μm particle size

Column oven: 50° C.

Mobile phase A—acetonitrile

Mobile phase B—40 mM ammonium formate, pH 3.45

Mobile phase C—24 mM formic acid pH 2.65 (not used)

Mobile phase D—HPLC grade H₂O

Gradient:

| Time | A % | B % | C % | D % | Flow | Curve |
|---|---|---|---|---|---|---|
| 0.00 | 2.0 | 40.0 | 0.0 | 58.0 | 0.500 | 1 |
| 2.00 | 2.0 | 40.0 | 0.0 | 58.0 | 0.500 | 6 |
| 10.00 | 20.0 | 40.0 | 0.0 | 40.0 | 0.500 | 6 |
| 11.00 | 90.0 | 0.0 | 0.0 | 10.0 | 0.500 | 6 |
| 15.00 | 90.0 | 0.0 | 0.0 | 10.0 | 0.500 | 6 |
| 16.00 | 2.0 | 40.0 | 0.0 | 58.0 | 0.500 | 6 |
| 25.00 | 2.0 | 40.0 | 0.0 | 58.0 | 0.500 | 6 |

The mass spectrometer was operated using ESI (electrospray sample introduction). The instrument was operated in negative mode using the following settings:

Polarity ESCi−

| | |
|---|---|
| Capillary (kV) | 2.80 |
| Corona (uA) | 0.30 |
| ES Cone (V) | 35.00 |
| Extractor (V) | 3.00 |
| RF Lens (V) | 0.5 |
| Source Temperature (° C.) | 150 |
| Desolvation Temperature (° C.) | 450 |
| Cone Gas Flow (L/Hr) | 20 |
| Desolvation Gas Flow (L/Hr) | 700 |
| Ion Energy 1 | 0.5 |
| Multiplier (V) | 650 |

A VICI 2-position valve (Valco Instruments) was used to divert flow to waste to avoid introducing sulfuric acid into the mass spectrometer. The valve was controlled by the MassLynx operating software.

Extraction ion chromatograms for succinic acid (117 amu) and levulinic acid (115 amu) were used for relative comparisons between samples.

Size exclusion analyses were performed using a Waters 2695 Alliance Separations module connected to a Waters 2996 PDA. Two Agilent PLgel3 μm 300×7.5 mm columns were connected in series. The mobile phase was tetrahydrofuran and the flow was 1.0 mL/min. The column oven was set to 30° C. Response was monitored at 300 nm.

### Description of Reagents

I: Composition: 5.47% levulinic acid, 2.25% formic acid, 0.165% glucose, 31% sulfuric acid, 0.1-2% soluble oligomers of unknown composition, 3.0% of filterable solid particles, and the remainder was water. I was derived from the acidic decomposition of High Fructose Corn Syrup (HFCS-42, Cornsweet® 42, ADM, Inc.) description of HFCS-42: 36.7% glucose, 30.3% fructose, 1.7% maltose, and 0.3% maltotriose. The mixture was filtered before use to remove solids. LC-MS ratio of levulinic acid to succinic acid was 220.9

### Example 6

15.2 g of crude levulinic acid mixture (I) was added into a 3-necked round bottom flask containing a magnetic stirrer, a reflux condenser, and a thermocouple. 9.8 g of 65% HNO₃ was slowly added into the mixture. It was heated to 40° C. for 70 minutes, and the solution had changed color from dark black into reddish-brown color and the solution was transparent. The reaction was heated to 65° C. After 25 minutes, the solution became lighter in color and the reddish-brown gas of NO₂ was noticeable. A sample was taken, which showed the presence of succinic acid by HPLC (peak area=155425). The reaction was stopped after 280 minutes, and a sample was taken out of the reactor and analyzed by HPLC. The sample showed the presence of succinic acid (peak area=131253, 0.81% by mass). LC-MS confirmed the compound as succinic acid. LC-MS ratio of levulinic acid to succinic acid was 0.012, indicating a much higher ratio of succinic acid to levulinic acid compared to the starting composition, I. Acetic acid was also produced as a co-product.

### Example 7

18 g of crude levulinic acid mixture (I) was added into a 3-necked round bottom flask containing a magnetic stirrer, a reflux condenser, and a thermocouple. 0.017 g of RuCl₃×H₂O was added as a catalyst. In a separate 20 mL scintillation vial, a solution containing 2.275 g of oxone and 10.297 g of water was made. After complete dissolution of the oxone, the oxone solution was added into the crude levulinic acid mixture over 5 minutes of addition time. The solution bubbled, and the solution color began to lighten from black to dark brown. The solution was stirred for 90 minutes at room temperature, then heated to 40° C. and stirred for 150 minutes. Then, the mixture was stirred for 150 minutes at 60° C. The color continued to lighten over the reaction time. The mixture became transparent. Succinic acid was produced, and confirmed by LC-MS. By LC-MS, the ratio of levulinic acid to succinic acid concentrations was 35.3, indicating a much higher ratio of succinic acid to levulinic acid compared to the starting composition, I.

### Example 8

15 g of crude levulinic acid mixture (I) was added into a 3-necked round bottom flask containing a magnetic stirrer, a reflux condenser, and a thermocouple. The flask was immersed in an ice bath. In a separate 20 mL scintillation vial, a solution containing 3 g of DI water and 0.5 g of sodium nitrite was made. After complete dissolution of the sodium nitrite, the sodium nitrite solution was added into the crude levulinic acid mixture over 5 minutes of addition time. The solution bubbled, and brown NO₂ gas formed. The solution color began to lighten from black to orange. The solution was stirred for 180 minutes at room temperature. The color continued to lighten over the reaction time. The mixture became transparent. Succinic acid was produced, and confirmed by LC-MS. By LC-MS, the ratio of levulinic acid to succinic acid concentrations was 68.3, indicating a much higher ratio of succinic acid to levulinic acid compared to the starting composition, I.

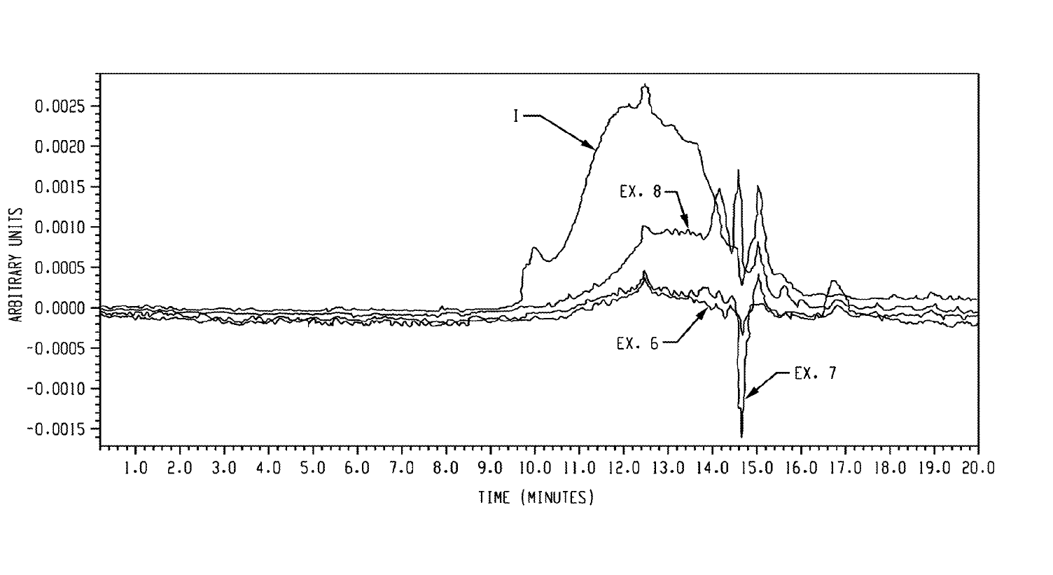

Surprisingly, it was found that this new process made succinic acid and also reduced the amount of undesirable high molecular weight oligomers as measured by SEC. FIG. 1 shows the SEC chromatograms for composition I and Examples 6-8. Examples 6-8 show a significant reduction in the amount of soluble oligomer impurities that were present in the initial composition I. The reduction in these impurities will aid in the downstream purification steps to purify succinic acid and levulinic acid.

As used herein, the singular forms “a,” “an,” and “the” are intended to include the plural forms as well, unless the context clearly indicates otherwise. The term “or” means “and/or.” The endpoints of all ranges directed to the same component or property are inclusive of the endpoint and independently combinable, except when the modifier “between” is used. The modifier “about” used in connection with a quantity is inclusive of the stated value and has the meaning dictated by the context (e.g., includes the degree of error associated with measurement of the particular quantity). A “combination” is inclusive of blends, mixtures, alloys, reaction products, and the like.

In general, the compositions or methods can alternatively comprise, consist of, or consist essentially of, any appropriate components or steps disclosed. The compositions can additionally, or alternatively, be formulated so as to be devoid, or substantially free, of any components, materials, ingredients, adjuvants, or species, or steps used in the prior art compositions or that are otherwise not necessary to the achievement of the function and/or objectives of the present claims.

Unless otherwise defined, all terms (including technical and scientific terms) used have the same meaning as commonly understood by one of ordinary skill in the art to which this invention belongs. Compounds are described using standard nomenclature. Any position not substituted by any indicated group is understood to have its valency filled by a bond as indicated, or a hydrogen atom. A dash (“-”) that is not between two letters or symbols is used to indicate a point of attachment for a substituent. For example, —OH is attached through carbon of the carbonyl group. “Alkyl” means a straight or branched chain saturated aliphatic hydrocarbon having the specified number of carbon atoms.

While stereochemistry of the various compounds is not explicitly shown, it is to be understood that this disclosure encompasses all isomers.

All cited patents, patent applications, and other references are incorporated by reference in their entirety.

While the invention has been described with reference to exemplary embodiments, it will be understood by those skilled in the art that various changes can be made and equivalents can be substituted for elements thereof without departing from the scope of the claims. In addition, many modifications can be made to adapt a particular situation or material to the teachings of the invention without departing from the essential scope thereof. Therefore, it is intended that the claims not be limited to the particular embodiment disclosed as the best mode contemplated for carrying out this invention, but that the invention will include all embodiments falling within the scope of the appended claims.

What is claimed is:

1. A method for producing a dicarboxylic acid from biomass, wherein the method comprises
obtaining from biomass a crude composition comprising, along with at least 1.0 wt. % of impurities comprising biomass residues, a ketocarboxylic acid having the following structure 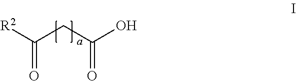
wherein R² is C₁-C₆ alkyl and a=0-3;
reacting the crude composition to oxidize the ketocarboxylic acid in the presence of an oxidizing agent to produce the corresponding dicarboxylic acid in which the R2 is replaced by an OH; and
optionally purifying and drying the dicarboxylic acid to obtain a solid composition comprising greater than 95 wt. % of dicarboxylic acid.

2. The method of claim 1, wherein the oxidizing agent is nitric acid and optionally a catalyst comprising vanadium pentoxide, and optionally in the further presence of a metallic nitrite.

3. The method of claim 1, wherein the crude composition is an acidic hydrolysate of the biomass and comprises at least 5 wt. % water.

4. The method of claim 1, wherein the hydrolysate is obtained employing concentrated sulfuric acid.

5. (canceled)

6. The method of claim 1, further comprising removing char particles from the crude composition prior to oxidizing the ketocarboxylic acid to the corresponding dicarboxylic acid.

7. The method of claim 1, wherein the oxidizing agent comprises nitric acid and further 0.1 to 1.0% by weight, calculated on the basis of nitric acid, of sodium nitrite is added.

8. The method of claim 1, wherein R² is C₁-C₆ alkyl and a=1-3.

9. (canceled)

10. (canceled)

11. The method of claim 1, wherein the ketocarboxylic acid is obtained from a biomass conversion in the presence of an acid catalyst comprising a Brønsted-Lowry acid, a Lewis acid, or a combination that comprises at least one of the foregoing catalysts.

12. The method of claim 1, wherein the crude composition comprising the ketocarboxylic acid comprises at least 5 wt. % of impurities comprising biomass residue, solid humins, extraction solvent, reaction by-products, reactants, and combinations thereof.

13. (canceled)

14. The method of claim 1, wherein the impurities comprise furfural, formic acid, furfuryl alcohol, hydroxymethyl furfural, angelica lactone, acetic acid, methanol, humin, soluble oligomer pre-cursors, polysaccharide, saccharide, extraction solvent, or a combination comprising at least one of the foregoing impurities.

15. The method of claim 1, wherein the ketocarboxylic acid is levulinic acid.

16. The method of claim 1, wherein the dicarboxylic acid is succinic acid.

17. (canceled)

18. (canceled)

19. A method for the production of succinic acid or succinic anhydride from biomass, the method comprising obtaining a crude composition comprising levulinic acid from biomass and converting the levulinic acid in the composition into succinic acid or succinic anhydride by a process as defined in claim 1.

20. (canceled)

21. (canceled)

22. (canceled)

23. A method for producing succinic acid from biomass, wherein the method comprises
obtaining from biomass, in the presence of an acid catalyst, a hydrolysate that is a crude composition comprising, along with at least 1.0 wt. % of impurities comprising biomass residues and at least 5 wt. % water, levulinic acid having the following structure: 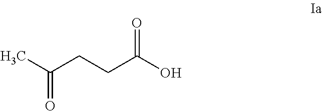
reacting the crude composition to oxidize the levulinic acid, in the presence of an oxidizing agent, to succinic acid, wherein the crude composition is heated in a continuous reaction system to a temperature in the range of from 20° C. to 100° C. for less than four hours followed by quenching; and
optionally purifying and drying the succinic acid to obtain a solid composition comprising greater than 95 wt. % of succinic acid.

24. The method of claim 23, further comprising filtering char particles from the crude composition before reacting the crude composition to oxidize the levulinic acid.

25. The method of claim 23, wherein the hydrolysate is obtained from biomass employing sulfuric acid.

26. The method of claim 23, wherein the impurities comprise furfural, formic acid, furfuryl alcohol, hydroxymethyl furfural, angelica lactone, acetic acid, methanol, humin, polysaccharide, saccharide, extraction solvent, or a combination comprising at least one of the foregoing impurities.

27. The method of claim 23, wherein the oxidizing agent is selected from a permanganate, a hypochlorite, oxygen, ozone, OXONE®, nitric acid, nitric oxide, sodium nitrite, a peroxide, or combinations thereof.

28. (canceled)

29. (canceled)

30. The method of claim 23, wherein a catalyst is used in combination with the oxidizing agent.

31. The method of claim 30, wherein the catalyst is a metal containing catalyst comprising platinum, palladium, ruthenium, copper, cobalt, vanadium, tungsten, iron, silver, manganese, or gold.

32. (canceled)

33. (canceled)
